(19) United States
(12) Patent Application Publication
(10) Pub. No.: US 2019/0240406 A1
(43) Pub. Date: Aug. 8, 2019

(54) ARTIFICIAL PANCREAS

(71) Applicants: CENTRE NATIONAL DE LA RECHERCHE SCIENTIFIQUE, Paris (FR); ÉCOLE CENTRALE DE NANTES, Nantes (FR); CENTRE HOSPITALIER UNIVERSITAIRE DE NANTES, Nantes Cedex 1 (FR); UNIVERSITÉ DE NANTES, Nantes (FR); CONSEJO NACIONAL DE INVESTIGACIONES CIENTIFICAS Y TECNICAS (CONICET), Caba (AR); INSERM (INSTITUT NATIONAL DE LA SANTÉ ET DE LA RECHERCHE MÉDICALE), Paris Cedex 13 (FR)

(72) Inventors: Claude MOOG, Nantes (FR); Nicolas MAGDELAINE, Nantes (FR); Santiago RIVADENEIRA, Santa Fe (AR); Lucy CHAILLOUS, Saint-Sébastien-sur-Loire (FR); Michel KREMPF, Nantes (FR)

(73) Assignees: CENTRE NATIONAL DE LA RECHERCHE SCIENTIFIQUE, Paris (FR); ÉCOLE CENTRALE DE NANTES, Nantes (FR); CENTRE HOSPITALIER UNIVERSITAIRE DE NANTES, Nantes Cedex 1 (FR); UNIVERSITÉ DE NANTES, Nantes (FR); CONSEJO NACIONAL DE INVESTIGACIONES CIENTIFICAS Y TECNICAS (CONICET), Caba (AR); INSERM (INSTITUT NATIONAL DE LA SANTÉ ET DE LA RECHERCHE MÉDICALE), Paris Cedex 13 (FR)

(21) Appl. No.: 16/341,494

(22) PCT Filed: Oct. 13, 2017
(86) PCT No.: PCT/EP2017/076209

§ 371 Date: Apr. 12, 2019

Related U.S. Application Data
(60) Provisional application No. 62/407,723, filed on Oct. 13, 2016.

(51) Int. Cl. A61M 5/172; G16H 20/17

(52) U.S. Cl. CPC A61M 5/1723; A61M 2205/50; A61M 2230/201; G16H 20/17

(57) ABSTRACT

A method for controlling an insulin injection device and a system for delivering insulin in a patient in need thereof. The method includes the steps of defining a time interval; receiving the level of blood glucose; computing an insulin dose to be injected in the next time interval, and transmitting the computed insulin dose to be injected to the insulin injection device.

### FIELD OF INVENTION

The present invention relates to the field of instrumentation with relation to pancreas insufficiency, especially diabetes, more specifically type-I diabetes. Indeed, this invention proposes a new method and a new system implementing a novel control strategy for compensating hyperglycemia in a fasting scenario, while ensuring positivity of the control and no hypoglycemic episodes. The novel control strategy is also dedicated to hybrid closed-loop where the patient chooses his bolus at meal time.

### BACKGROUND OF INVENTION

Insulin was discovered almost 100 years ago. Until today, it is the only treatment for type-1 diabetes. This treatment consists in multiple daily insulin injections. Basal-bolus schemes are widely used. Bolus Advisors are designed to help patient to compute bolus doses.

Functional Insulin Therapy

Same food, same injection, at same time of the day was an option for type-1 diabetes treatment but it was not very satisfactory. Functional insulin therapy is an educational program that helps patient to compute insulin injections. It defines tools as the insulin sensitivity factor (ISF) and the carbo ratio (CR). These tools, empirically estimated from clinical protocols are used to compute insulin boluses depending on Blood Glucose (BG) level, Blood Glucose target, carbohydrates (CHO) in the meal and previous boluses.

The definitions of these tools are the following:
ISF, also known as correction factor (CF), is the Blood Glucose drop caused by 1 unit of rapid-acting insulin;
CR, is the amount of CHO that compensates the glycemic drop caused by 1 unit of rapid-acting insulin.

ISF and CR allow to compute meal and correction boluses:
The Correction Bolus U_{BG}, depending on the patient's CF, BG level and BG target is:

$${U}_{BG} = \frac{{BG}_{level} - {BG}_{target}}{CF} \qquad ( 1 )$$

The Meal Bolus U_{Carb} depends on the patient's CR and the amount of carbohydrates CHO in the meal:

$${U}_{Carb} = \frac{CHO}{CR} \qquad ( 2 )$$

These tools are used in everyday life by diabetic patients to compute the insulin bolus given by:

U_{Bol}=U_{BG}+U_{Carb} (3)

Nevertheless, patients have difficulties in computing the correct insulin doses because:
CF might vary with the time of the day, physical activity, stress or illness;
CR varies according to meal composition.

Hence, every meal turns into a stressful math problem for most type-1 diabetics.

The Bolus Wizard

Nowadays, glucometers and insulin pump include a Bolus Wizard. Physicians inform these calculators with individualized values of CR, CF or Blood Glucose Target according to the time of the day. Thus diabetic patients only have to enter the estimated amount of CHO to obtain insulin dose recommendations.

However, one of the most common error is over-correcting a post-meal rise in Blood Glucose. It occurs when the amount of insulin that is still active in the body is not properly taken into account. This amount is called Insulin On Board (IOB). Most Bolus Wizard include the IOB to avoid hypoglycemia. The bolus is computed as:

U_{Bol}=U_{BG}+U_{Carb}−IOB (4)

IOB is a function of the Duration of Insulin Action (DIA) and the amount of previous boluses. IOB is computed in different ways according to the different Bolus Wizards. Nonetheless, incorrect estimation of DIA induces mismatch in the IOB and insulin injection. As a consequence, hypoglycemia occurs when DIA is underestimated while overestimation of DIA leads to hyperglycemia. Determination of individualized DIA remains a critical point.

Since the past 50 years, closed-loop control of Blood Glucose in type-1 diabetes, the so-called artificial pancreas (AP), remains a challenge. In 1977, the Biostator was the first realization of an artificial pancreas. Many families of controllers were designed, among which are the Proportional-Integrate-Derivative (PID), PID with insulin feedback, Biohormonals, sliding modes, fuzzy-logic and model-predictive controllers (MPC). The latter became popular because they included constraints on the control and safety algorithms. Nowadays, closed-loop clinical trials are conducted for inpatients and outpatients.

Still, ambulatory artificial pancreas systems are not available because many improvements are needed. Among them, for MPC control algorithms:
the prediction horizon has to be extended;
the accuracy of predictions given by the model has to be improved;
the individualization of the controller requires an engineering expert work;

Consequently, there is still a need to provide an artificial pancreas that could guarantee positivity of the control while avoiding hypoglycemic episodes.

### SUMMARY

According to a first aspect, the present invention relates to a method for delivering insulin in a patient in need thereof.

The method comprising:
determining a time interval;
measuring the level of blood glucose of the patient at the final endpoint of each time interval;
using a processor for computing a global insulin injection rate to be injected at the final endpoint of each time interval, said computing of the injection rate taking into account patient's own parameters such as for example blood glucose and insulin on board; and
delivering said computed global insulin injection rate to be injected at the final endpoint of each time interval to the patient.

According to one embodiment, said time interval ranges from 1 millisecond to 3 hours, from 0.1 second to 1 hour or from 1 second to 15 minutes.

According to one embodiment, the computed global insulin injection rate comprises a constant insulin injection rate such as basal rate and a variable insulin injection rate.

According to one embodiment, the global insulin injection rate to be injected at the final endpoint of each time interval is computed for tuning the velocity of decrease of glycemia of the patient without reaching hypoglycemia levels.

According to one embodiment of the method,
at least one window of time comprising a plurality of time interval is defined;
for a window of time, a total amount of insulin to be injected is determined; and
the global insulin injection rate at the final endpoint of each time interval is function of the parameters of blood glucose and insulin on board, as computed by the processor.

According to one embodiment, said window of time is a period of time higher than 12 hours, can be 24-72 hours and can also last several months to several years.

According to a second aspect, the invention relates to a computer program product, comprising a non-transitory tangible computer readable medium having a computer readable program code embodied therein, which is adapted to be executed to implement a method for delivering insulin. The method comprising:
defining a time interval;
measuring the level of blood glucose of the patient at the final endpoint of each time interval;
using a processor for computing a global insulin injection rate to be injected at the final endpoint of each time interval, said computing of the injection rate taking into account the parameters of blood glucose and insulin on board; and
delivering said computed global insulin injection rate to be injected at the final endpoint of each time interval to the patient.

According to a third aspect, the invention further relates to a system for delivering insulin, the system comprising a computer program product according to the second aspect of the present invention; an insulin pump; and a means for measuring the level of blood glucose of the patient in a patient body such as a glucose sensor or continuous glucose measurement; wherein the system is capable to execute the method according to the first aspect of the present invention. According to one embodiment, said means for continuous glucose measurement is connected to said computer program product.

According to a fourth aspect, the present invention also relates to a computer-implemented method for controlling an insulin injection device of a diabetic user, comprising iteratively the steps of:
determining a time interval Tₛ;
receiving the blood glucose level;
computing an insulin dose to be injected u(nTₛ) in the next time interval; and
transmitting the computed insulin dose to be injected to an insulin injection device of a diabetic user.

The computed insulin dose to be injected u(nTₛ) is computed according to the formula:

u(nTₛ)=k_{d}×ũ(nTₛ)+Tₛ×U_{Bas}.

In this formula, U_{Bas} is the diabetic user's specific basal insulin injection rate and Tₛ×U_{Bas} corresponds to the basal dose which is always injected in order to get closer to a real pancreas.

By “iteratively”, it had to understood that the steps of receiving, computing and transmitting (and optionally the step of determining) are continuously repeated.

ũ(nTₛ) is a correction insulin dose which correspond to the insulin dose to be injected in order to reach to a blood glucose level of reference. This correction insulin dose is computed as ũ(nTₛ)=u_{BG}(nTₛ)−IOB(nTₛ) wherein u_{BG}(nTₛ) is the insulin dose needed to reach the blood glucose level x₁(nTₛ) to a blood glucose level target x₁_{_{ref}} without considering previous insulin injections; and IOB(nTₛ) is the insulin dose still active in the body of the diabetic user.

In this way, the computed insulin dose to be injected u(nTₛ) is always positive and does not need to be set at zero if the blood glucose level.

The k_{d} coefficient is a tuning parameter strictly positive and inferior or equal to 1. When k_{d} is strictly inferior to 1, the method injects only a part of the needed dose in order to spread in the time the insulin dose to be injected. This tuning parameter acts like a safety parameter. Indeed, if the blood glucose level becomes lower than expected because of an error in parameters or because of a physical activity of the diabetic user, the shifting in time of a part of the insulin injection permits to avoid hypoglycemia to the diabetic user.

According to one embodiment, the insulin dose needed to reach the blood glucose level x₁(nTₛ) to a blood glucose level target x₁_{_{ref}} without considering previous insulin injections is computed as:

$${u}_{BG} ( {nT}_{s} ) = \frac{{x}_{1} ( {nT}_{s} ) - {x}_{{1}_{ref}}}{{θ}_{2}}$$

wherein x₁_{_{ref}} is the target blood glucose level; and θ₂ is the diabetic user's specific insulin sensitivity factor.

According to one embodiment, IOB(Tₛ) is computed as:

IOB(nTₛ)=θ₃×(x₂(nTₛ)+x₃(nTₛ))
wherein
   x₂(nTₛ) is the plasma insulin rate;
   x₃(nTₛ) is the subcutaneous insulin rate; and
   θ₃ is the diabetic user's specific insulin response time.

According to one embodiment, the insulin dose to be injected u(nTₛ) comprises a proportional component to the blood glucose level x₁(nTₛ), a derivative component to the blood glucose level x₁(nTₛ) and a second derivative component to the blood glucose level x₁(nTₛ).

According to one embodiment, x₂(nTₛ) is computed as x₂(nTₛ)=U_{Bas}−1/θ₂×\dot{x}₁(nTₛ). According to one embodiment, x₃(nTₛ) is computed as x₃(nTₛ)=x₂(nTₛ)−θ₃×(\ddot{x}₁(nTₛ))/θ₂. In said embodiments, \dot{x}₁(nTₛ) is the time derivative of the blood glucose level x₁(nTₛ) and \ddot{x}₁(nTₛ) is the second time derivative of the blood glucose level x₁(nTₛ).

According to an alternative embodiment, x₂(nTₛ) and x₃(nTₛ) are determined by an observer. Such observer can be an algorithm or a device which measures the amount of insulin injected since a predetermined time.

According to one embodiment, the parameter k_{d} is strictly positive and strictly inferior to 1. According to one embodiment, the parameter k_{d} is strictly positive and inferior or equal to 0.99, 0.95, 0.90, 0.85 or 0.80 . . .

According to one embodiment, x₁_{_{ref}} ranges from 70 mg/L to 140 mg/L. According to one embodiment, the time interval Tₛ ranges from 1 millisecond to 3 hours, from 0.1 second to 1 hour or from 1 second to 15 minutes.

According to one embodiment, the method further comprises the step of computing a second insulin dose u_{Carb} to be injected when an actuator is activated, the second insulin dose u_{Carb} corresponding to the dose of insulin to be injected compensating a meal. According to one embodiment, the actuator is activated before, during or after a meal or when a meal is detected. According to another embodiment, the actuator is activated manually by the diabetic user. The latter corresponds to the so-called hybrid closed-loop.

According to a fifth aspect, the present invention further relates to a system for delivering insulin. The system comprises:
a processor comprising instructions to operate the computer-implemented method according to the fourth aspect of the present invention;
an insulin injection device; and
a sensor for measuring the blood glucose level of a diabetic user.

According to one embodiment, said sensor is connected to the processor in order to provide to said processor the blood glucose level x₁(nTₛ).

According to one embodiment, the processor comprising a processor device and at least one memory element associated with the processor, the at least one memory element storing processor-executable instructions that, when executed by the processor, perform a method of controlling delivery of insulin from insulin injection device to the body of the diabetic user according to the fourth aspect of the present invention.

The insulin injection device is controlled by the processor and is able to inject into the patient body the insulin rate during a time interval or the insulin dose at the end of each time interval computed by the processor with the method according to the fourth aspect of the present invention.

According to one embodiment, the insulin injection device comprising an insulin reservoir for insulin to be delivered from the insulin injection device to a body of a user.

In a sixth aspect, the invention relates to a closed-loop insulin infusion system comprising: a continuous glucose sensor that generates sensor data indicative of sensor glucose values for a user; and an insulin infusion device to receive the sensor data generated by the continuous glucose sensor, the insulin infusion device comprising: an insulin reservoir for insulin to be delivered from the insulin infusion device to a body of a user; a processor architecture comprising at least one processor device; and at least one memory element associated with the processor architecture, the at least one memory element storing processor-executable instructions that, when executed by the processor architecture, perform a method of controlling closed-loop delivery of insulin from the insulin reservoir to the body of the user, the method comprising:
initiating a closed-loop operating mode of the insulin infusion device; in response to initiating the closed-loop operating mode, obtaining a most recent sensor glucose value for the user;
computing a current insulin on board (IOB(nTₛ)) value that indicates an amount of active insulin in the body of the user;
determining an insulin dose to be injected u(nTₛ) during a predetermined time interval (Ts);
operating the insulin infusion device in a closed-loop mode to deliver insulin from the insulin reservoir to the body of the user in accordance with insulin dose to be injected that is determined, wherein the insulin dose to be injected represents an amount of insulin to be delivered during each time interval (Ts).

According to a seventh aspect, the invention relates to a method for delivering insulin in a patient in need thereof, the method comprising the steps of:
defining a time interval;
measuring the level of blood glucose x₁(t) of the patient at the final endpoint of each time interval;
using a processor for computing a global insulin injection rate uᵢ(t) to be injected during the next time interval or at the final endpoint of each time interval; and
delivering said computed global insulin injection rate uᵢ(t) to be injected during the next time interval or at the final endpoint of the next time interval to the patient.

In said aspect of the invention, uᵢ(t)=k×ũ₁(t)+U_{Bas}.

U_{bas} is a constant patient's specific basal insulin rate; k is a tuning parameter strictly positive and inferior or equal to 1; ũ₁(t) is a variable insulin injection rate computed as:

$${\tilde{u}}_{1} ( t ) = \frac{{x}_{1} ( t ) - {x}_{1 ref}}{{θ}_{2}} - {θ}_{3} {x}_{2} ( t ) - {θ}_{3} {x}_{3} ( t ) ;$$

wherein
x₂(t) is computed as x₂(t)=U_{Bas}−1/θ₂×\dot{x}₁(t);
x₃(t) is computed as x₃(t)=x₂(t)−θ₃×(\ddot{x}₁(t))/θ₂;
x_{1ref} is a blood glucose level target;
\dot{x}₁(t) is the time derivative of the blood glucose level x₁(t);
\ddot{x}₁(t) is the second time derivative of the blood glucose level x₁(t);
θ₂ is the patient's specific insulin sensitivity factor;
θ₃ is the diabetic user's specific insulin response time.

According to one embodiment, the steps of measuring the level of blood glucose, using a processor for computing global insulin injection rate and delivering said computed global injection rate are continuously executed at each time interval, optionally during a predetermined window of time.

According to one embodiment, the parameter k is strictly positive and strictly inferior to 1. According to another embodiment, the parameter k is equal to 1.

The insulin dose delivered at each time interval (according to the fourth aspect) equals the insulin rate (according to the eighth aspect) times the time interval. Thus k_{d} defines as:
k_{d}=1−e^{(-k·T}^{ₛ}⁾ where k is in rad/s and k_{d} is dimensionless

According to an eighth aspect, the present invention relates to a computer program product comprising instructions which, when the program is executed by a computer, causes the computer to carry out the steps of:
receiving a level of blood glucose x₁(t) of a patient;
computing a global insulin injection rate uᵢ(t) to be injected;
transmitting the computed global insulin injection rate uᵢ(t) to an insulin injection device;

wherein uᵢ(t)=k×ũ₁(t)+U_{Bas}; and U_{Bas} is a constant patient's specific basal insulin rate, k is a tuning parameter strictly positive and inferior or equal to 1 and ũ₁(t) is a variable insulin injection rate computed as:

$${\tilde{u}}_{1} ( t ) = \frac{{x}_{1} ( t ) - {x}_{1 ref}}{{θ}_{2}} - {θ}_{3} {x}_{2} ( t ) - {θ}_{3} {x}_{3} ( t ) ;$$

and further wherein:
x₂(t) is computed as x₂(t)=U_{Bas}−1/θ₂×\dot{x}₁(t);
x₃(t) is computed as x₃(t)=x₂(t)−θ₃×(\ddot{x}₁(t))/θ₂;
x_{1ref} is a blood glucose level target;
\dot{x}₁(t) is the time derivative of the blood glucose level x₁(t);
\ddot{x}₁(t) is the second time derivative of the blood glucose level x₁(t);
θ₂ is the patient's specific insulin sensitivity factor;
θ₃ is the diabetic user's specific insulin response time.

According to one embodiment, the parameter k is strictly positive and strictly inferior to 1. According to another embodiment, the parameter k is equal to 1.

According to a ninth aspect, the invention also relates to a system for delivering insulin, the system comprising: a computer program product according to the invention; an insulin pump; and a means for measuring the level of blood glucose of the patient in a patient body such as a glucose sensor or continuous glucose measurement; wherein the system is capable to execute the method according to the present invention.

According to a tenth aspect, the invention relates to a method for controlling an insulin injection device of an user, comprising iteratively the steps of:
determining a time interval Tₛ;
receiving the blood glucose level x₁(nTₛ);
computing an insulin dose to be injected u(nTₛ) in the next time interval;
wherein the insulin dose to be injected u(nTₛ) comprises at least:
a first term being a function of the comparison between the received blood glucose level x₁(nTₛ) and a predefined blood glucose level target x₁_{_{ref}} in a preliminary step of the method;
a second term, the second term being an estimated value of the insulin dose still active in the body IOB(nTₛ) of the user;
the second term being superior or equal to the first term at each iteration of the method.

The advantage of the feature “the second term being superior or equal to the first term at each iteration of the method” is to preserve the positivity of the computed insulin dose. In this way, the computed insulin dose to be injected is always positive and does not need to be set at zero in case of an insulin dose to be injected become negative. The positivity of the command ensure a security to the user.

In one embodiment, the first and the second terms of the insulin dose to be injected is a function of a corrective factor inferior or equal to 1, the corrective factor being configured to adapt the duration of the injection to a predefined duration reference.

In one embodiment, the first and the second terms of the insulin dose to be injected is a linear function of the corrective factor.

The advantage of this embodiment is to inject only a fraction of the calculated insulin dose which the user theoretically needs to reach the blood glucose level target. This tuning parameter acts like a safety parameter. Indeed, if the blood glucose level becomes lower than expected because of an error in parameters or because of a physical activity of the diabetic user, the shifting in time of a part of the insulin injection permits to avoid hypoglycemia to the diabetic user.

In one embodiment, the insulin dose to be injected comprises a third term calculated on at least one specific injection rate of a predefined user profile. In one embodiment, said third term is constant on each iteration. In one embodiment, said third term is constant along a predefined time comprising a plurality of adjacent iterations.

The advantage of said third term is to provide a basal rate of insulin to mimic the behavior of a health human pancreas and to ensure that at least a minimum amount of insulin is injected at each iteration.

In one embodiment, the insulin dose to be injected is a function of at least one of the following predefined user profile parameters: a specific insulin response time; and/or a specific insulin sensitivity factor. The advantage of said embodiment is to use some coefficient which is usually handle by the user and the doctor. Furthermore, said coefficient are not an average or a statistical but can easily be measured precisely for each diabetic user.

In one embodiment, the first term is a function of the specific insulin sensitivity factor and/or the second term is a function of both the specific insulin response time and the specific insulin sensitivity factor.

In one embodiment, the first term is a function of:

$$\frac{{x}_{1} ( {nT}_{s} ) - {x}_{{1}_{ref}}}{{θ}_{2}}$$

In one embodiment, the second term is a function of:

θ₃×(x₂(nTₛ)+x₃(nTₛ));

wherein:
x₂(nTₛ) is the plasma insulin rate; and
x₃(nTₛ) is the subcutaneous insulin rate.

In one embodiment, x₂(nTₛ) is computed as x₂(nTₛ)=U_{Bas}−1/θ₂×\dot{x}₁(nTₛ) or x₂(nTₛ) is a function of U_{Bas}−1/θ₂×\dot{x}₁(nTₛ).

In one embodiment, x₃(nTₛ) is computed as x₃(nTₛ)=x₂(nTₛ)−θ₃×(\ddot{x}₁(nTₛ))/θ₂ or x₃(nTₛ) is a function of x₂(nTₛ)−θ₃×(\ddot{x}₁(nTₛ))/θ₂.

\dot{x}₁(nTₛ) is the time derivative of the blood glucose level x₁(nTₛ) and \ddot{x}₁(nTₛ) is the second time derivative of the blood glucose level x₁(nTₛ). U_{Bas} is an user's specific basal insulin injection rate.

In one embodiment, the insulin dose to be injected comprises at least a proportional component to the blood glucose level, a derivative component to the blood glucose level and a second derivative component to the blood glucose level.

In one embodiment, the insulin dose to be injected does not comprise a term which is function of an integral of the blood glucose level.

In one embodiment, the insulin dose to be injected comprises a fourth term being a function of a second insulin dose corresponding to the dose of insulin to be injected compensating a predefined ingested quantity of glucose by the user. The advantage is to take into account an amount of glucose ingested by the user during the day or during the method.

In one embodiment, the corrective factor is positive or strictly positive and strictly inferior to 1.

In one embodiment, the step of computing an insulin dose is executed by a calculator.

In one embodiment, the method is implemented by a computer.

In one embodiment, the method further comprises the step of transmitting the computed insulin dose to be injected to the insulin injection device.

According to a eleventh aspect, the invention further relates to a system for delivering insulin, said system comprising:
a processor comprising instructions to operate the method according to the tenth aspect of the present invention;
an insulin injection device; and
a sensor for measuring the blood glucose level of an user.

In one embodiment, the system further comprises a transmitter to transmit data from the sensor to the processor and to transmit data from the processor to the insulin injection device.

In one embodiment, the system further comprises an interface configured to define the at least one following parameter: a specific insulin response time; and/or a specific insulin sensitivity factor; and/or a specific user basal insulin injection rate.

### DETAILED DESCRIPTION

This invention proposes a method, a computer program and a system implementing a state feedback control law, derived from functional insulin therapy, in order to compensate high glycemia levels during a fasting period or in a hybrid closed-loop. This state feedback control law computes basal-boluses injections, provides predictions on glucose dynamics using a long-term model, guarantees positivity of the control, and allows avoiding hypoglycemic episodes.

The system of the invention also offers the advantage that it is easy to set-up.

According to the invention, the tuning of the control law is individualized simply using patient's own parameters such as for example the correction factor and the duration of insulin action. Thanks to the use of the patient's own parameters, the tuning is readily understandable to physicians, pump manufacturers, and patients themselves.

Insulin on Board

In this section, the model of the glucose-insulin dynamics used in the present invention is presented. Then it is established that the Insulin on Board can be computed as a combination of the states.

A long-term model of the glucose-insulin dynamics for type-1 diabetes is used in the present invention. Considering a fasting scenario, x₁ is the BG, x₂ and x₃ are the plasma and subcutaneous compartment insulin rate [U/min], respectively. The input uᵢ is the insulin injection rate [U/min]. θ₁ is the net balance between the endogenous glucose production and the insulin independent consumption, θ₂ is the ISF and θ₃ is the time constant of the insulin subsystem related to the DIA. The model is:

$$\begin{matrix}{\dot{x}}_{1} = {θ}_{1} - {θ}_{2} {x}_{2} , & ( 5 ) \\ {\dot{x}}_{2} = - \frac{1}{{θ}_{3}} {x}_{2} + \frac{1}{{θ}_{3}} {x}_{3} , & ( 6 ) \\ {\dot{x}}_{3} = - \frac{1}{{θ}_{3}} {x}_{3} + \frac{1}{{θ}_{3}} {u}_{i} . & ( 7 )\end{matrix}$$

Notice that all the states xᵢ and the control variable uᵢ represent physiological entities, therefore all are positive variables.

The insulin injection rate uᵢ is mostly the sum of a basal rate and boluses: uᵢ=U_{Bas}+u_{bol}. Thus the states x₂ and x₃ can also be written as sums:

x₃=x₃_{_{Bas}}+x₃_{_{bol}}=x₃_{_{Bas}}+\tilde{x}₃, (8)

x₂=x₂_{_{Bas}}+x₂_{_{bol}}=x₂_{_{Bas}}+\tilde{x}₂. (9)

In fasting period, the correct basal insulin rate is established when glycemia is maintained constant. The equilibrium values x_{2Bas} and x_{3Bas} are:

$${U}_{Bas} = \frac{{θ}_{1}}{{θ}_{2}} ⇒ {x}_{{3}_{Bas}} = {x}_{{2}_{Bas}} = \frac{{θ}_{1}}{{θ}_{2}} , \qquad ( 10 )$$

and by using Eqs. (5) and (10) glycemia dynamics becomes:

$${\dot{x}}_{1} = {θ}_{1} - {θ}_{2} {x}_{2} = {θ}_{1} - {θ}_{2} ( {x}_{{2}_{Bas}} + {\tilde{x}}_{2} ) , \\ {\dot{x}}_{1} = {θ}_{1} - {θ}_{2} ( \frac{{θ}_{1}}{{θ}_{2}} + {\tilde{x}}_{2} ) , \\ {\dot{x}}_{1} = - {θ}_{2} {\tilde{x}}_{2} . \qquad ( 11 )$$

A physiological definition of Insulin on Board is either: the units of insulin from previous boluses that are still active in the body, or the amount of insulin in the subcutaneous and the plasma compartments after boluses. According to the first definition, the state representation and the input u_{bol}, the IOB can be written as:

IOB(t)=∫₀^{t}(u_{bol}(τ)−\tilde{x}₂(τ))dτ. (12)

Now, merging Eqs. (6) and (7):

θ₃(\tilde{\dot{x}}₃+\tilde{\dot{x}}₂)=u_{bol}−\tilde{x}₂. (13)

Considering that no bolus was made before t=0, one gets \tilde{x}₃(0)=0 and \tilde{x}₂(0)=0. Then with (12) and (13):

IOB(t)=θ₃∫₀^{t}(\tilde{\dot{x}}₃(τ)+\tilde{\dot{x}}₂(τ))dτ,

IOB(t)=θ₃(\tilde{x}₃(t)+\tilde{x}₂(t)). (14)

which agrees with the second physiological definition.

Another equivalent interpretation is:

IOB(t)=∫ₜ^{∞}\tilde{x}₂(τ)dτ, (15)

when considering only previous boluses, the assumption is made that u_{bol}(τ)=0 for any τ≥t. Thus

∫₀^{∞}u_{bol}(τ)dτ=∫₀^{∞}\ddot{x}₂(τ)dτ=∫₀^{t}u_{bol}(τ)dτ

IOB(t)=∫ₜ^{∞}\ddot{x}₂(τ)dτ=∫₀^{t}(u_{bol}(τ)−\tilde{x}₂(τ))dτ. (16)

Integrating Eq. (11) and comparing it with (15)

θ₂IOB(t)=x₁(t)−x_{∞}. (17)

which reads as the foreseen drop of glycemia level due to on board insulin, in other words, IOB provides long-term prediction on glycemia.

Control Law Design

According to this invention, a new control law called ‘Dynamic Bolus Calculator’ (DBC) is introduced. In one embodiment, the DBC is based on the correction bolus formula (4) with U_{Carb}=0 (i.e. considering no meal),

$$\begin{matrix}Eq . ( 14 ) and ( 17 ) , with ISF = CF = {θ}_{2} \text{:} \\ \tilde{u} ( t ) = {U}_{BG} ( t ) - IOB ( t ) , \\ ⇒ \tilde{u} ( t ) = \frac{{x}_{1} ( t ) - {x}_{1 ref}}{{θ}_{2}} - {θ}_{3} ( {\tilde{x}}_{3} ( t ) + {\tilde{x}}_{2} ( t ) ) & \\ Defining {\tilde{x}}_{1} ( t ) = {x}_{1} ( t ) - {x}_{1 ref} & \\ \tilde{u} ( t ) = {\tilde{u}}_{1} ( t ) = \frac{{\tilde{x}}_{1} ( t )}{{θ}_{2}} - {θ}_{3} {\tilde{x}}_{2} ( t ) - {θ}_{3} {\tilde{x}}_{3} ( t ) . & ( 18 )\end{matrix}$$

In one embodiment, the invention consists to use the equation (18) in continuous. The θ₂ and θ₃ parameters are provided to the computer program and are tools usually handled by the patient. In consequence, an advantage is the method according to the present invention is personalized and very simple to be applied to different diabetic users.

According to one embodiment, the computed global insulin injection rate comprises a constant insulin injection rate such as basal rate and a variable insulin injection rate.

The global injection rate uᵢ(t) will be the state feedback modulating ũ₁(t) the constant insulin injection rate U_{Bas}:

uᵢ(t)=U_{Bas}+ũ₁(t). (19)

Thus, with (6), (7), (11) and (18), the following closed loop will be studied:

\tilde{\dot{x}}(t)=A\ddot{x}(t)+Bũₖ(t), \ddot{x}(0)=\dot{x}₀. (20)

ũₖ(t)=Fₖ\tilde{x}, (21)

where ũₖ is defined as ũₖ=kũ. This feedback defines an entire family of DBC controllers, which are part of this invention. The matrices A, B, and Fₖ are

$$A = ( \begin{matrix}0 & - {θ}_{2} & 0 \\ 0 & - \frac{1}{{θ}_{3}} & \frac{1}{{θ}_{3}} \\ 0 & 0 & - \frac{1}{{θ}_{3}}\end{matrix} ) , B = ( \begin{matrix}0 \\ 0 \\ \frac{1}{{θ}_{3}}\end{matrix} ) , {F}_{k} = k ( \begin{matrix}\frac{1}{{θ}_{2}} & - {θ}_{3} & - {θ}_{3}\end{matrix} ) .$$

An interesting property of this family of controllers is that the total quantity of injected insulin does not depend on k:

∫₀^{∞}ũₖ(τ)dτ=∫₀^{∞}ũ₁(τ)dτ=ũ₁(0). (22)

This allows to stretch the input trajectory and to keep constant the total quantity of insulin injected, and k just tunes the velocity of decrease of glycemia without falling into hypoglycemia levels, as shown hereafter.

Input/State Positivity

In this section, important properties as stability and positivity of the closed-loop trajectories are addressed. It is proven that this feedback generates a positive control, which ensures the positivity of, \tilde{x}₁ that is x₁(t)≥x_{1ref}.

In medical terms, this property is a guaranty of no hypoglycemic episodes.

According to Eqs. (20-21), the closed-loop system reads as:

$$\dot{\tilde{x}} ( t ) = ( A + {BF}_{k} ) \tilde{x} ( t ) = \tilde{A} \tilde{x} ( t ) , \\ \dot{\tilde{x}} ( t ) = ( \begin{matrix}0 & - {θ}_{2} & 0 \\ 0 & - \frac{1}{{θ}_{3}} & \frac{1}{{θ}_{3}} \\ \frac{k}{{θ}_{2} {θ}_{3}} & - k & - k - \frac{1}{{θ}_{3}}\end{matrix} ) \tilde{x} ( t ) , \qquad ( 23 )$$

which is a stable system with eigenvalues

$${λ}_{1} = {λ}_{2} = - \frac{1}{{θ}_{3}}$$

and λ₃=−k, for all k>0.

The positivity of the input/state trajectories, i.e. ũₖ(t)≥0 and \tilde{x}(t)≥0 ∀t≥0, is discussed through the notion of positively invariant sets.
Definition 1: Given a dynamical system \dot{x}−Dx. x∈ [symbol] ⁿ, and a trajectory x(t, x₀), where x₀ is a initial condition, a nonempty set M ⊆ [symbol] ⁿ is a positive invariant set if ∀x₀ ∈ M ⇒ x(t, x₀)∈M ∀t≥0.
Definition 2: If G∈ [symbol] ^{r×n} then M(G) denotes the polyhedron

M(G)={x∈ [symbol] ⁿ|Gx≥0}. (24)

Proposition 1: The polyhedral set M(G) is a positively invariant set for the system of Definition 1 if and only if there exists a Metzler matrix

H∈ [symbol] ^{r×r}, i.e. Hᵢⱼ≥0 for i≠j, such that:

GD−HG=0. (25)

Note that the polyhedron defined by the positive orthant in R³ is not a positively invariant set for the system (23) since in this case G=I and D=Ã, and H must be Ã; however the latter matrix is not Metzler.

In order to find the maximal invariant set, the system (23) is transformed to its Jordan form, i.e.

ż(t)=P⁻¹ÃPz(t)=Jz(t). (26)

where \tilde{x}=Pz, and

$$J = ( \begin{matrix}- \frac{1}{{θ}_{3}} & 1 & 0 \\ 0 & - \frac{1}{{θ}_{3}} & 0 \\ 0 & 0 & - k\end{matrix} ) \qquad ( 27 )$$

through the matrix of change of coordinates

$$P = ( \begin{matrix}{θ}_{2} {θ}_{3} & {θ}_{2} {θ}_{3}^{2} & 1 \\ 1 & 0 & \frac{k}{{θ}_{2}} \\ 0 & {θ}_{3} & \frac{k ( 1 - k {θ}_{3} )}{{θ}_{2}}\end{matrix} ) . \qquad ( 28 )$$

Notice that the positive orthant in the new coordinates is a positively invariant set because the matrix J is Metzler.

The state trajectories \tilde{x}(t) in z-coordinates are

$$\begin{matrix}{\tilde{x}}_{1} ( t ) = {θ}_{2} {θ}_{3} {e}^{\frac{- t}{{θ}_{3}}} ( {z}_{1} ( 0 ) + ( {θ}_{3} + t ) {z}_{2} ( 0 ) ) + {e}^{- kt} {z}_{3} ( 0 ) & ( 29 ) \\ {\tilde{x}}_{2} ( t ) = {e}^{\frac{- t}{{θ}_{3}}} ( {z}_{1} ( 0 ) + {tz}_{2} ( 0 ) ) + {e}^{- kt} \frac{k}{{θ}_{2}} {z}_{3} ( 0 ) , & ( 30 ) \\ {\tilde{x}}_{3} ( t ) = {e}^{\frac{- t}{{θ}_{3}}} {θ}_{3} {z}_{2} ( 0 ) - {e}^{- kt} \frac{k ( k {θ}_{2} - 1 )}{{θ}_{2}} {z}_{3} ( 0 ) , & ( 31 )\end{matrix}$$

and the control trajectory becomes

$${\tilde{u}}_{k} ( t ) = {e}^{- kt} \frac{{k ( k {θ}_{3} - 1 )}^{2}}{{θ}_{2}} {z}_{3} ( 0 ) , \qquad ( 32 )$$

It verifies that

$$\frac{{k ( k {θ}_{3} - 1 )}^{2}}{{θ}_{2}} {z}_{3} = {\tilde{u}}_{k} = k ( \frac{1}{{θ}_{2}} {\tilde{x}}_{1} - {θ}_{3} ( {\tilde{x}}_{2} + {\tilde{x}}_{3} ) ) . \qquad ( 33 )$$

Now, the property (22) can be proved. Consider the integral of Eq. (32)

$${\int }_{0}^{∞} {\tilde{u}}_{k} ( τ ) d τ = - \frac{{( k {θ}_{3} - 1 )}^{2}}{{θ}_{2}} {e}^{- kt} {z}_{3} ( 0 ) {\vert}_{0}^{∞} = \frac{{( k {θ}_{3} - 1 )}^{2} {z}_{3} ( 0 )}{{θ}_{2}} .$$

Replacing z₃ from Eq. (33) in the latter equation

$${\int }_{0}^{∞} {\tilde{u}}_{k} ( τ ) d τ = \frac{1}{{θ}_{2}} {\tilde{x}}_{1} ( 0 ) - {θ}_{3} ( {\tilde{x}}_{2} ( 0 ) + {\tilde{x}}_{3} ( 0 ) ) = {\tilde{u}}_{1} ( 0 ) .$$

As the control trajectory Eq. (32) is an exponential function depending on k, that allows us to stretch the trajectory ensuring that the same quantity of insulin is administered for all k>0.

The following theorem restates the positivity of the first orthant in z-space, but in the x-coordinates.

Theorem 1: Consider the sets M₁= [symbol] \tilde{x}∈ [symbol] ³|\tilde{x}≥0}, and

$${M}_{2} = \{ \tilde{x} ∈ {ℝ}^{3} \vert {k ( \frac{1}{{θ}_{2}} {\tilde{x}}_{1} - {θ}_{3} ( {\tilde{x}}_{2} + {\tilde{x}}_{3} ) )}^{′} \geq 0 \} .$$

The maximal positively invariant polyhedron of system (23) is

M=M₁ ∩ M₂. (34)

Proof: It is clear that the condition x(0)≥0 is necessary but it does not ensure that the \tilde{x}-trajectories remain positive for t>0, because the matrix Ã is not Metzler. However, the set {\tilde{x}∈ [symbol] ³|\tilde{x}≥0} contains any positively invariant set, i.e.

M ⊂ M₁.

In z-coordinates, the positively invariant set is the first orthant in [symbol] ³, and by Eq. (32), z₃ is proportional to ũ, therefore ũ≥0 is a necessary condition but not sufficient. Then

M ⊂ M₂.

Now, using Definition 2, the polyhedron {Gx≥0} where

$$G = ( \begin{matrix}1 & 0 & 0 \\ 0 & 1 & 0 \\ 0 & 0 & 1 \\ \frac{k}{{θ}_{2}} & - k {θ}_{3} & - k {θ}_{3}\end{matrix} ) , \qquad ( 35 )$$

characterizes the positive invariant set for the system (23), and translates the positive orthant in z to x-coordinates. That is verified using Proposition 1 with the following H-matrix

$$H = ( \begin{matrix}- \frac{1}{{θ}_{3}} & 0 & {θ}_{2} & \frac{{θ}_{2}}{k {θ}_{3}} \\ 0 & - \frac{1}{{θ}_{3}} & \frac{1}{{θ}_{3}} & 0 \\ 0 & 0 & - \frac{1}{{θ}_{3}} & \frac{1}{{θ}_{3}} \\ 0 & 0 & 0 & - k\end{matrix} ) , \qquad ( 36 )$$

And D=Ã. As this Proposition is a necessary and sufficient condition, then the set M=M₁ ∩ M₂ is the maximal positively invariant polyhedron for system (23).

According to Definition 1, the nonempty set M ⊆ [symbol] ³ is the positively invariant polyhedron of the system (22) controlled by Eq. (21), that is, if the system starts inside M, it will remains there for any t>0. As the insulin subsystem is indeed positive, the condition to ensure the positivity can be summarized as ũ≥0.

From a medical point of view, the positivity of the input ensures that [symbol] ≥0, i.e. guaranties the exclusion of hypoglycemia episodes:

(x₁≥x₁_{_{ref}}, ∀t≥0):

Moreover, positivity of the control stands in agreement with the management of insulin injection.

Nonetheless, the eigenvalues of the insulinemia subsystem

$$( - \frac{1}{{θ}_{3}} )$$

are not modified by the control law. Consequently the performance of the closed-loop depends on the patient's θ₃ parameter.

Robustness

Robustness is a decisive issue as it ensures that the controller will work safely on the non-nominal diabetic patient.

According to one embodiment, the processor for computing further defines a reference level of Blood Glucose; and wherein at the final endpoint of each time interval, the global insulin injection rate is corrected, taking into account the gap between the measured level of Blood Glucose and a reference level of Blood Glucose.

Delay Margin

Delays are well known to destabilize closed-loops. Here, it is studied the robustness with respect to delays that were not taken into account in the model. These delays appear naturally in the closed-loop of the artificial pancreas. Analyzing the state feedback (23), the target loop transfer is given by:

$${L}_{Target} = {F}_{k} \cdot {( sI - A )}^{- 1} \cdot B \\ {L}_{Target} = \frac{k}{s} \qquad ( 37 )$$

The phase margin of L_{Target} is

$$\frac{π}{2}$$

at pulsation ωₖ=k. This leads to a delay margin

$${M}_{r} = \frac{π}{2 {ω}_{k}} , \\ {M}_{r} = \frac{π}{2 k} ,$$

which is the maximum added delay that does not destabilize the loop. For instance, setting Mr=25 min resolves

k=kᵣ≅10⁻³ rad/s.

Performance by Regulation

The closed-loop shows that it is possible to reject disturbance acting as an output step, but not as a ramp. In the case of a ramp disturbance, the speed error will be:

$$ϵ = \frac{slope}{k}$$

Parameters Uncertainties

Because the behavior of the organism of the user could be different of the nominal behavior, the calculated bolus is not delivered in one dose but is spread in time. In this case, the steep fall in Blood Glucose rate is limited.

With the factor kᵣ which ensures stability of the closed-loop for delays lower than 25 minutes, robustness with respect to parameters uncertainties is studied. The state feedback is:

$${\hat{F}}_{{k}_{r}} = {k}_{r} [ \frac{1}{{\hat{θ}}_{2}} - {\hat{θ}}_{3} - {\hat{θ}}_{3} ] , \qquad ( 38 )$$

Where \hat{θ}ₗ are estimates of the model parameters θᵢ. Considering the state feedback A+B\hat{F}ₖ_{ᵣ}, the target loop transfer is given by:

$$\hat{L} = {{\hat{F}}_{{k}_{r}} ( sI - A )}^{- 1} \cdot B \\ \hat{L} = {k}_{r} \frac{\frac{1}{{θ}_{3}^{3}} ( \frac{{θ}_{2}}{{θ}_{2}} + s {\hat{θ}}_{3} + {θ}_{3} {\hat{θ}}_{3} s ( s + \frac{1}{{θ}_{3}} ) )}{{s ( s + \frac{1}{{θ}_{3}} )}^{2}} \qquad ( 39 )$$

With the Nyquist criterion, FIG. 1 shows that stability is ensured even with great parameters uncertainties. Moreover, the delay margin is still good as it is equal to 12 min at worst for \hat{θ}_{2,3}=50%×θ_{2,3}.

### BRIEF DESCRIPTION OF THE FIGURES

FIG. 1 represents L_{Target} and \hat{L} with 50%×θ_{2,3}≤\hat{θ}_{2,3}≤200%×θ_{2,3}.

FIG. 2 represents state feedback Fₖᵣ with delay Tᵣ not taken into account and with well-known model parameter (\hat{θ}=θ).

FIG. 3 represents Dawn Phenomenon: open and closed-loop with state feedback Fₖᵣ.

FIG. 4 represents closed-loop with state feedback Fₖᵣ and CF underestimated.

FIG. 5 represents Dynamic Bolus Calculator control law with Adult01 from UVA/Padova T1DMS wearing Generic pump and Dexcom 70 CGM device. Estimated parameters: \hat{θ}₁=1.6 mg/dl/min, \hat{θ}₂=75 mg/dl/U, \hat{θ}₃=38 min.

FIG. 6 represents the glycemia level of a diabetic patient (above) and the amount of insulin injection (below) during time. Two models are represented: the model with one bolus of insulin injection (named “bolus” on the graph) and the model according to one embodiment of the present invention wherein the insulin injection is continuously calculated and injected according to the present invention and wherein the k coefficient is strictly inferior to 1 (“spread bolus”) and wherein the loop is closed at t=30 min.

FIG. 7 represents an enlargement of the graph on FIG. 6.

### RESULTS

The following simulations are conducted under meal-free scenarios. The reference is set to 100 mg/dl and loop is closed at t=60 minutes.

The patient's parameters, derived from a real patient, are θ₁=0.85 mg/dl/min, θ₂=70 mg/dl/U and θ₃=62 min.

Closed-Loop with Delay

All CGM devices introduce some delay due to the physio-logical time lag between blood glucose and interstitial glucose concentration.

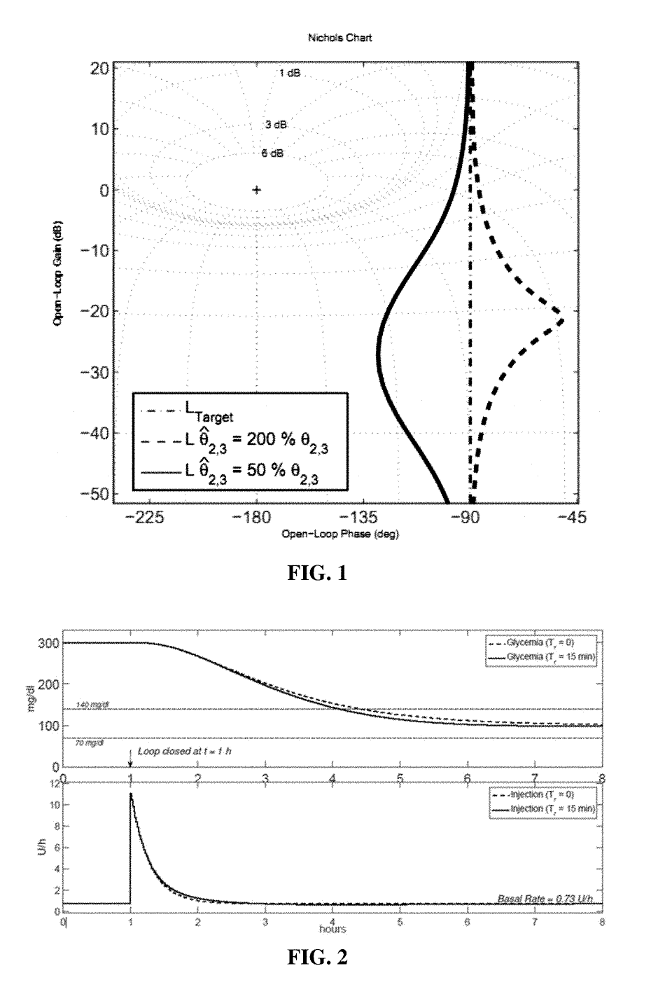

FIG. 2 illustrates the closed-loop (23) with a delay Tᵣ added to the state \tilde{x}₁. The state feedback Fₖᵣ uses the delayed output \tilde{x}₁(t−Tᵣ) and the current states \tilde{x}₂(t) and \tilde{x}₃(t).

At the beginning (t=0) BG=300 mg/dl, IOB=0 U.

As argued in section “Input/State Positivity”, in the nominal case (Tr=0):
the control remains positive ũₖᵣ≥0 (as uᵢ≥U_{Bas});
the state remains positive \tilde{x}₁≥0 (as x₁≥x_{ref}), there is no hypoglycemic episode;
the speed of return in euglycemia depends on θ₃ as explained in section “Input/State Positivity”.

When Tᵣ=15 min
loop remains stable as expected (see part “Delay Margin”);
there is no hypoglycemic episode;
although it was no demonstrated in case of delay ∫ũₖ_{ᵣ}|_{τ}_{ᵣ}₌₀=∫üₖ_{ᵣ}|_{τ}_{ᵣ}₌₁₅=2.86 U which corresponds to the injection advised by a bolus wizard at t=1 h with respect to Eq. (4).

The Dawn Phenomenon

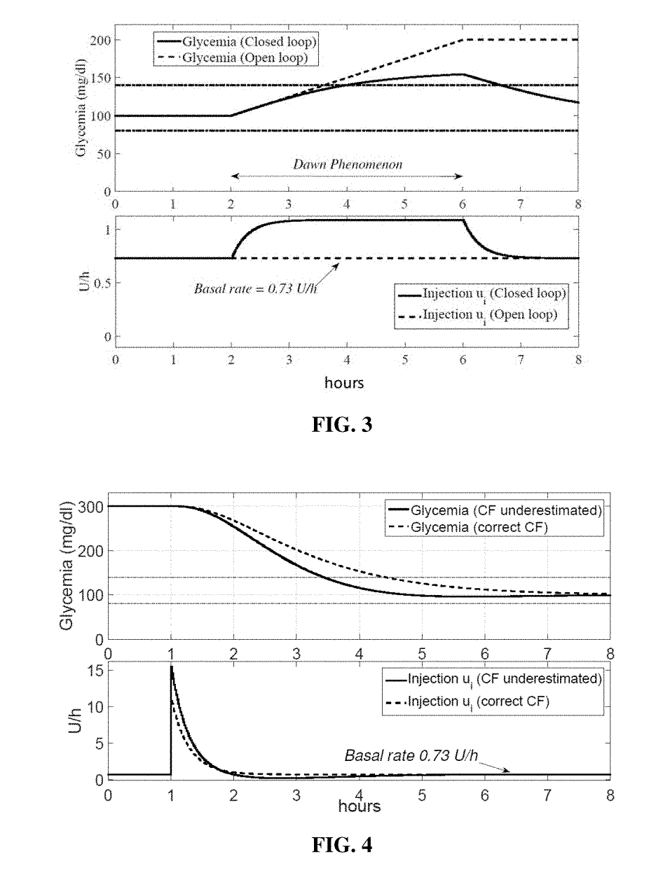

The dawn phenomenon is an increase in blood glucose in the night due to surge in hormone secretion. FIG. 3 illustrates the closed-loop (23) using the state feedback Fₖᵣ, with the dawn phenomenon modeled by a ramp disturbance on the output of 25 mg/dl/h between 2 and 6 a.m.

In open loop, under constant basal rate, the dawn phenomenon brings glycemia to 200 mg/dl at 8 a.m.

In closed-loop:
the state feedback control law produces a temporary basal delivery rate;
as expected (see part “Performance by regulation”) the effect of the ramp disturbance;
and BG recovers euglycemia at 8 a.m.

Parameter Uncertainties

In this section uncertainty on CF will be addressed. Assuming that the patient has CF of 70 mg/dl/U, the worst case is considered: CF is underestimated (\hat{θ}₂=70%×θ₂=50 mg/dl/U).

The initial glycemia is 300 mg/dl and the target is 100 mg/dl. In open loop, this would involve dramatic consequences as the computed bolus ((300−100)/50=4 U) should lower the glycemia by CF×4 U=280 mg/dl and lead the patient to severe hypoglycemia (BG above 20 mg/dl).

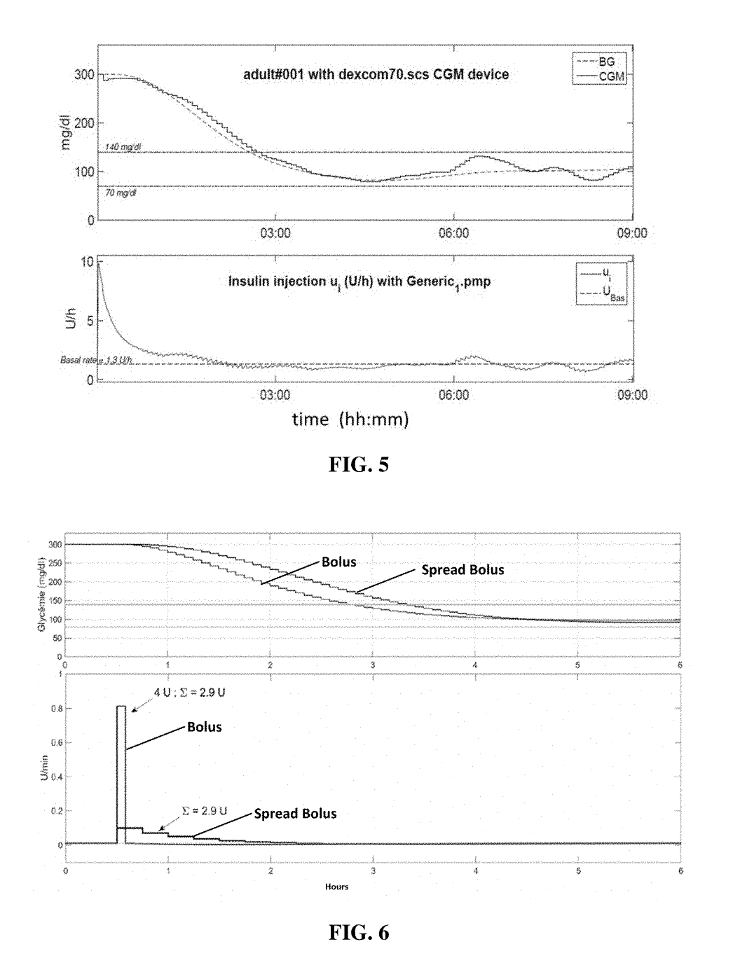

FIG. 4 shows high safety of the closed-loop as despite an underestimation of the CF, the glycemia reaches target with no hypoglycemia (the minimum BG is 96 mg/dl).

UVA/Padova T1DM Simulator

The distributed version of the UVA/Padova has been approved by the Food and Drug Administration as a preclinical testing platform for control algorithm. With several virtual patients, it also includes models of pumps and CGM devices. This simulator is used to demonstrate the safety and efficiency of the DBC control algorithm. The parameters (\hat{θ}₁, \hat{θ}₂ and \hat{θ}₃) of the virtual patient are identified from a previous scenario. The initial BG is 300 mg/dl. At t=0 the loop is closed. The virtual patient uses a Generic pump (increase step is 0.05 U) and a CGM device (Dexcom 70) which introduces delay and noise and has a sampling time of 5 minutes. The reference has been set to 110 mg/dl.

FIG. 5 shows a good performance of the closed-loop as:
BG is into euglycemia at 2 h 3;
there is no hypoglycemia (minimum BG is 82 mg/dl).

Robustness of the closed-loop is also demonstrated as:
the model of the virtual patient is non-nominal;
the CGM devices introduces delays, noise and a sampling time of 5 min;
the pump has a minimum delivery rate step of 0.05 U;
there was no saturation of the controller.

### CONCLUSION

Individualization of the controller and accurate prediction for MPC algorithm are still an open problem in the artificial pancreas project. Here, a novel closed-loop is developed. With a structure derived from bolus advisors, this controller is simply tuned with individualized characteristics of the patient (CF and DIA). Thus it is immediately comprehensive to physicians, and patients.

The main feature of this control law is to ensure the positivity of trajectories. This guarantees that the glycemia remains greater than its reference, at least in the nominal case and allows the controller to cope with the positivity constraint of the insulin injection.

As in practice there are some delays, and parameter uncertainties, a robustness analysis is added.

Finally, through simulations the performance of the loop is assessed, for the nominal case, and for a more realistic scenario, the UVA/Padova simulator was used to implement this.

As the dynamics of the insulinemia subsystem are not modified, a control law is envisioned to accelerate the response. Also, it is necessary to tackle the problem with meals and a long term scenario. The good performance obtained with the simulator encourages to propose clinical trials in this subject.

### EXAMPLE 1

### Comparison Between One Single Bolus Dose and a Portioned Bolus Dose

One degree of freedom in the tuning of the controller is the time in which a bolus is delivered. One can choose to deliver a bolus in one single dose or to partition said single dose. The last point is a pledge of security. FIG. 6 shows a simulation wherein patient parameters are known. The loop is closed after a duration of 30 minutes. One will notice that a “Bolus” instruction generates one single bolus dose then delivers the basal bolus dose (basal rate). The “Spread Bolus” works with a control period of 15 minutes and delivers 77% of the bolus in 1 hour.

FIG. 6 shows a simulation when the patient compensatory value is not well entered. The patient, here, has a compensatory value equal to 70 mg/dl/U and the controller works with a wrong value of 50 mg/dl/U. Thus, the generated bolus dose is of 4 U, which leads the glycemia to a final value of 300−4*70 that corresponds to a calculated value of 20 mg/dl.

However, the controller observes the deviation between the measured glycemia value and the target glycemia value, given by the IOB and corrects at the next instruction by retracting a part of the basal dose (1.1 U for 4 hours). The global value remains strictly positive. As mentioned, the “spread bolus” instruction provides a pledge of security. Indeed, 3.5 U are injected in 1 h 45 then 0.6 U are retracted of the basal dose.

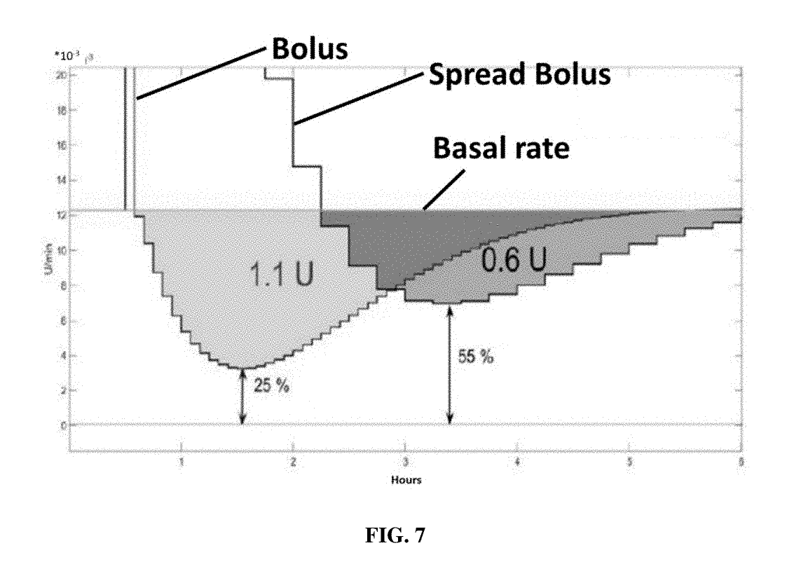

One can observe on the enlargement on FIG. 7, with the “bolus” instruction that the corrector response to the overdose is performed at the second clock tick in order to reach its maximal effect (75% of the retracted basal dose) one hour after the bolus release. Therefore, the “spread bolus”, expressed by a coefficient k_{d} strictly inferior to 1, allows keeping better margins (45% of the basal value retracted in the worst case).

1. A method for controlling an insulin injection device of a user, comprising iteratively the steps of:
determining a time interval Tₛ;
receiving the blood glucose level x₁(nTₛ);
computing an insulin dose to be injected u(nTₛ) in the next time interval;
wherein the insulin dose to be injected u(nTₛ) comprises at least:
a first term being a function of the comparison between the received blood glucose level x₁(nTₛ) and a predefined blood glucose level target x₁_{_{ref}} in a preliminary step of the method;
a second term, the second term being an estimated value of the insulin dose still active in the body IOB(nTₛ) of the user;
the second term being superior or equal to the first term at each iteration of the method.

2. The method according to claim 1, wherein the first and the second terms of the insulin dose to be injected u(nTₛ) is a function of a corrective factor (k_{d}) inferior or equal to 1, the corrective factor (k_{d}) being configured to adapt the duration of the injection to a predefined duration reference.

3. The method according to claim 2, wherein the first and the second terms of the insulin dose to be injected u(nTₛ) is a linear function of the corrective factor (k_{d}).

4. The method according to claim 1, wherein the insulin dose to be injected u(nTₛ) comprises:
a third term calculated on at least one specific injection rate of a predefined user profile.

5. The method according to claim 1, wherein the insulin dose to be injected u(nTₛ) is a function of at least one of the following predefined user profile parameters:
a specific insulin response time θ₃; and/or
a specific insulin sensitivity factor θ₂.

6. The method according to claim 1, wherein the first term is a function of:
$$\frac{{x}_{1} ( nTs ) - {x}_{{1}_{ref}}}{{θ}_{2}}$$
wherein θ₂ is a specific insulin sensitivity factor.

7. The method according to claim 1, wherein the second term is a function of:
θ₃×(x₂(nTₛ)+x₃(nTₛ))
wherein
θ₃ is a specific insulin response time;
x₂(nTₛ) is the plasma insulin rate; and
x₃(nTₛ) is the subcutaneous insulin rate.

8. The method according to claim 7, wherein
x₂(nTₛ) is computed as x₂(nTₛ)=U_{Bas}−1/θ₂×\dot{x}₁(nTₛ); and
x₃(nTₛ) is computed as x₃(nTₛ)=x₂(nTₛ)−θ₃×(\ddot{x}₁(nTₛ))/θ₂;
wherein \dot{x}₁(nTₛ) is the time derivative of the blood glucose level x₁(nTₛ) and \ddot{x}₁(nTₛ) is the second time derivative of the blood glucose level x₁(nTₛ), and further wherein U_{Bas} is a user's specific basal insulin injection rate and θ₂ is a specific insulin sensitivity factor.

9. The method according to claim 1, wherein the insulin dose to be injected u(nTₛ) comprises at least a proportional component to the blood glucose level x₁(nTₛ), a derivative component to the blood glucose level x₁(nTₛ) and a second derivative component to the blood glucose level x₁(nTₛ).

10. The method according to claim 1, wherein the insulin dose to be injected u(nTₛ) does not comprise a term which is a function of an integral of the blood glucose level x₁(nTₛ).

11. The method according to claim 1, wherein the insulin dose to be injected u(nTₛ) comprises:
a fourth term being a function of a second insulin dose u_{Carb} corresponding to the dose of insulin to be injected compensating a predefined ingested quantity of glucose by the user.

12. The method according to claim 2, wherein the corrective factor (k_{d}) is positive and strictly inferior to 1.

13-14. (canceled)

15. The method according to claim 1, said method further comprising the step of:
transmitting the computed insulin dose to be injected u(nTₛ) to the insulin injection device.

16-17. (canceled)

18. A computer-implemented method for controlling an insulin injection device of a user, comprising iteratively the steps of:
determining a time interval Tₛ;
receiving the blood glucose level x₁(nTₛ);
computing an insulin dose to be injected u(nTₛ) in the next time interval;
wherein u(nTₛ)=k_{d}×ũ(nTₛ)+Tₛ×U_{Bas} wherein:
k_{d} is a tuning parameter strictly positive and inferior or equal to 1;
ũ(nTₛ) is a correction insulin dose computed as ũ(nTₛ)=u_{BG}(nTₛ)−IOB(nTₛ); wherein
u_{BG}(nTₛ) is the insulin dose needed to reach the blood glucose level x₁(nTₛ) to a blood glucose level target x₁_{_{ref}} without considering previous insulin injections; and
IOB(nTₛ) is the insulin dose still active in the body of the user; and
U_{Bas} is the user's specific basal insulin injection rate.
transmitting the computed insulin dose to be injected u(nTₛ) to the insulin injection device.

19-38. (canceled)

39. A computer program comprising instructions for the implementation of steps of a method according to claim 1 when the program is executed by a computer.

40. A system for delivering insulin, the system comprising:
a processor comprising instructions to operate the method according to claim 1;
an insulin injection device; and
a sensor for measuring the blood glucose level of a user.

41. The system according to claim 40 further comprising an interface configured to define the at least one following parameter:
a specific insulin response time θ₃; and/or
a specific insulin sensitivity factor θ₂; and/or
a specific user basal insulin injection rate U_{Bas}.

42. A system for delivering insulin, the system comprising:
an insulin injection device;
a sensor for measuring the blood glucose level of a user;
a processor comprising instructions configured to iteratively operate steps of:
determining a time interval Tₛ;
receiving the blood glucose level x₁(nTₛ);
computing an insulin dose to be injected u(nTₛ) in the next time interval;
wherein the insulin dose to be injected u(nTₛ) comprises at least:
a first term being a function of the comparison between the received blood glucose level x₁(nTₛ) and a predefined blood glucose level target x₁_{_{ref}} in a preliminary step of the method;
a second term, the second term being an estimated value of the insulin dose still active in the body IOB(nTₛ) of the user;
the second term being superior or equal to the first term at each iteration.

43. The system according to claim 42, wherein the first and the second terms of the insulin dose to be injected u(nTₛ) is a function of a corrective factor (k_{d}) inferior or equal to 1, the corrective factor (k_{d}) being configured to adapt the duration of the injection to a predefined duration reference.

44. The system according to claim 42, wherein the insulin dose to be injected u(nTₛ) is a function of at least one of the following predefined user profile parameters:
a specific insulin response time θ₃; and/or
a specific insulin sensitivity factor θ₂.
